(19) United States
(12) Patent Application Publication
(10) Pub. No.: US 2024/0390699 A1
(43) Pub. Date: Nov. 28, 2024

(54) VULVAR STERILIZATION DRYER FOR WOMEN

(71) Applicant: Sung Hoon PARK, Seoul (KR)

(72) Inventor: Sung Hoon PARK, Seoul (KR)

(21) Appl. No.: 18/699,320

(22) PCT Filed: Sep. 5, 2022
(86) PCT No.: PCT/KR2022/013239

§ 371 Date: Apr. 8, 2024

(30) Foreign Application Priority Data
Oct. 12, 2021 (KR) 10-2021-0134628

(51) Int. Cl. A61N 5/06

(52) U.S. Cl. CPC A61N 5/0624; A61N 2005/0626; A61N 2005/0632; A61N 2005/0642; A61N 2005/0645; A61N 2005/0651

(57) ABSTRACT

The present invention relates to a vulvar sterilization dryer for women, and is configured to facilitate sterilization and drying of the vulva by making it possible to blow outside air or warm air around the anus while irradiating the vulva with an LED light source having a sterilization function that helps to prevent diseases by removing harmful bacteria.

### TECHNICAL FIELD

The present invention relates to a vulvar sterilization dryer for women, more specifically to a vulvar sterilization dryer for women that is capable of being configured to blow outside air or hot air toward the anal area, while irradiating the vulva with LED light sources having sterilization functions of removing harmful bacteria to thus prevent the occurrence of diseases, so that it is easy to sterilize and dry the vulva.

### BACKGROUND ART

Generally, heat therapy in a sitting position, which is based on a meridian system of traditional Oriental medicine, is one of representative Oriental medicines and folk remedies that applies hot heat to the reproductive organs, perineum, anus, and coccyx of men and women to achieve the activation of the organs related to them and accelerate blood circulation and metabolism, so that the functions of internal organs are activated.

In specific, hot heat is applied from the perineal blood as a representative essential point of surgery of the heat therapy in a sitting position to the reproductive organs, anus, and coccyx of men and women, simultaneously, so that heat stimulation is applied to the muscles, subcutaneous cells, micro nerves, blood vessels, spinal nerves, etc., which are related to the perineal blood, the reproductive organs, anus, and coccyx, to produce active bio-reactions of metabolism from the organs such as the heart, liver, stomach, lungs, intestines, and the like, thereby treating adult diseases such as hypertension, diabetes, cancers, heart diseases, womb diseases, coldness, gastrointestinal diseases, and the like and preventing incurable diseases from occurring.

Further, hot heat serves to dissolve some of fat accumulated in the abdomen area, saturated fat built up in blood vessels, thrombus (extravasated blood), and uric acid of fatigue factors, so that micro nerves and body fluids are activated and blood circulation and metabolism are accelerated to boost immunity, thereby optimizing the efficacy of the heat therapy in a sitting position.

Conventional heat therapy in a sitting position is classified into electric type heat therapy using electricity as a heat source and a toilet or jar type steam seat using steam generated from dried mugwort burnt.

In the case of the electric type heat therapy, metal or ceramic powder is heated with a power source to allow medicinal herbs to be burnt thereby, so that the heat or steam generated therefrom is applied to a user's body, and in the case of the toilet or jar type steam seat, mugwort or medicinal herb is burnt in the interior thereof, so that the heat generated therefrom is irradiated onto the reproductive organs, perineum, and anus. Recently, a product having a heating function, which is configured to mount a hot pad onto a woman's underwear, has been proposed to remove the coldness of the reproductive organ.

According to an electric local steamer as issued conventionally, coupling processes among electric parts and accessories such as a hot coil, a seat, a disc, and a latent heat material and the use of the latent heat material as a chemical product are needed in a process of generating hot heat, thereby causing many inconveniences in making and using the electric local steamer, and according to a jar type seat steamer attached to the underside of a seat, as issued conventionally, a burner is inserted into a jar to apply heat generated therefrom to the local area and the genital area. In this case, since the jar fixed to the underside of the seat is bulky, many inconveniences may occur in keeping and using the jar type seat steamer, thereby having limitations in popularly purchasing and using the jar type seat steamer.

In the case of the product having the steaming function that is configured to mount a hot pad onto a woman's underwear to remove the coldness of the reproductive organ, a bag (pocket) for accommodating the hot pad and a thermally conductive film connected to the bag apply stimulations onto the sensitive reproductive organ or anus of the woman, the hips are uncomfortably pressurized against the hot pad mounted onto the underwear, and thermally conductive heat is low at temperature, so that moxibustion, which causes the activation of the internal organs related to the reproductive organs, perineum, anus, and coccyx and the acceleration of the blood circulation, has no efficiency.

To solve such problems, a vulva fumigator for women (as disclosed in Korean Patent Application Laid-open No. 10-2020-0005147) has been filed and issued to the same applicant as the present invention.

The conventional vulva fumigator for women includes a housing open on one surface thereof to form a space portion therein and having a supply hole formed on top thereof to supply water, the housing being curved to cover the genital area and the anus when worn, a water storage part located on one side of the interior of the housing and having an inlet and an outlet adapted to store and supply the water through the supply hole, a controller fixed to the interior of the housing on one side of the water storage part under the water storage part and having a power supply part adapted to control the operations thereof according to the control of a smartphone having a Bluetooth function or the control of a remote controller, a fume supply part located on the other side of the water storage part under the water storage part in such a way as to operate according to the control of the controller to receive and vaporize the water stored in the water storage part, pass the vaporized water through a functional filter, and supply the water as fume, a spray member connected to the fume supply part by means of a supply pipe on which a check valve is mounted and having a plurality of spray nozzles for spraying the fume generated from the fume supply part into a user's genital area and anus, an irradiation part fixedly located on the front surface of the spray member in such a way as to expose the ends of the injection nozzles of the spray member to allow the fume to be sprayed and having LEDs mounted on the front surface thereof in such a way as to irradiate light emitted therefrom onto the genital area and the anus, the irradiation part being controllable in wavelength under the control of the controller, and a protection film having one end fixed to the edge of the housing and the other end protruding in one direction in such a way as to allow the housing to be brought into close contact with the user's skin when worn.

According to the conventional vulva fumigator for women, however, the fume is sprayed into the genital area and the anus through the fume supply part, while the light emitted from the LEDs is being irradiated onto the genital area and the anus, so that the anus and the area around the anus are not directly dried. In this case, to dry the anus and the anus area, it is actually impossible for a user (a disabled person, a pregnant woman, etc.) to use existing hair dryers. Therefore, there is a definite need to develop a vulvar sterilization dryer for women.

### DISCLOSURE OF THE INVENTION

### Technical Problems

Accordingly, the present invention has been made in view of the above-mentioned problems occurring in the related art, and it is an object of the present invention to provide a vulvar sterilization dryer for women that is capable of being configured to blow outside air or hot air toward the anal area, while irradiating the vulva with LED light sources having sterilization functions of removing harmful bacteria to thus prevent the occurrence of diseases, so that it is easy to sterilize and dry the vulva.

Other objects of the present invention will be obvious through the following description.

### Technical Solutions

To accomplish the above-mentioned objects, according to the present invention, there is provided a vulvar sterilization dryer for women, including: a curved plate-like base member 110 adapted to cover a user's genital area and anus and having an anus spray hole 110a formed on one side thereof; a controller 120 disposed on one side of one surface of the base member 110 and having an operating button 121, a power supply part 122, and a PCB 123 adapted to control operations thereof; an irradiation part 130 fixedly disposed onto the other surface of the base member 110 in such a way as to operate controllable in wavelength under the control of the controller 120 to allow light emitted from LEDs 132 to be irradiated onto the genital area and the anus; an anus spray part 140 disposed close to the controller 120 on one surface of the base member 110 in such a way as to operate under the control of the controller 120 to allow outside air or hot air to be sprayed into the anal area through the anus spray hole 110a; and a covering member 150 configured to allow one end thereof to be detachably coupled to one surface of the base member 110 to coveringly protect the controller 120 and the anus spray part 140 from the outside.

Further, the irradiation part 130 may include a curved plate-shaped substrate 131 having an anus communication hole 131a formed thereon in such a way as to communicate with the anus spray hole 110a, when fixed to the other surface of the base member 110, and the LEDs 132 located on the front surface of the substrate 131 in such a way as to emit light having a sterilization function of removing harmful bacteria to prevent occurrence of diseases toward the genital area and the anus.

Furthermore, the anus spray part 140 may include an anus spray housing 141 fixedly disposed onto one surface of the base member 110, an anus heater 142 located at one side of the interior of the anus spray housing 141 in such a way as to emit heat under the control of the controller 120, an anus blower 143 disposed close to the anus heater 142 at the inside of the anus spray housing 141 in such a way as to blow air toward the anus heater 142 under the control of the controller 120, and an anus supply pipe 144 having one end connected to the anus spray housing 141 and the other end connected to the anus spray hole 110a to allow the outside air or hot air to be supplied to the anus spray hole 110a.

### Advantageous Effects of the Invention

According to the present invention, the vulvar sterilization dryer for women is configured to blow outside air or hot air toward the anal area, while irradiating the vulva with the LED light sources having sterilization functions of removing harmful bacteria to thus prevent the occurrence of diseases, so that it is easy to sterilize and dry the vulva.

### BRIEF DESCRIPTION OF DRAWINGS

FIG. 1 is a perspective view showing top of a vulvar sterilization dryer for women according to the present invention.

FIG. 2 is a perspective view showing the underside of the vulvar sterilization dryer for women according to the present invention.

FIG. 3 is a sectional view showing the vulvar sterilization dryer for women according to the present invention.

FIG. 4 is a perspective view showing the interior of the vulvar sterilization dryer for women according to the present invention, from which a covering member is removed.

### BEST MODE FOR INVENTION

The present invention relates to a vulvar sterilization dryer for women including: a curved plate-like base member 110 adapted to cover a user's genital area and anus and having an anus spray hole 110a formed on one side thereof; a controller 120 disposed on one side of one surface of the base member 110 and having an operating button 121, a power supply part 122, and a PCB 123 adapted to control operations thereof; an irradiation part 130 fixedly disposed onto the other surface of the base member 110 in such a way as to operate controllable in wavelength under the control of the controller 120 to allow light emitted from LEDs 132 to be irradiated onto the genital area and the anus; an anus spray part 140 disposed close to the controller 120 on one surface of the base member 110 in such a way as to operate under the control of the controller 120 to allow outside air or hot air to be sprayed into the anal area through the anus spray hole 110a; and a covering member 150 configured to allow one end thereof to be detachably coupled to one surface of the base member 110 to coveringly protect the controller 120 and the anus spray part 140 from the outside, wherein the anus spray part 140 includes an anus spray housing 141 fixedly disposed onto one surface of the base member 110, an anus heater 142 located at one side of the interior of the anus spray housing 141 in such a way as to emit heat under the control of the controller 120, an anus blower 143 disposed close to the anus heater 142 at the inside of the anus spray housing 141 in such a way as to blow air toward the anus heater 142 under the control of the controller 120, and an anus supply pipe 144 having one end connected to the anus spray housing 141 and the other end connected to the anus spray hole 110a to allow the outside air or hot air to be supplied to the anus spray hole 110a.

### MODE FOR INVENTION

Hereinafter, an explanation of a vulvar sterilization dryer for women according to an embodiment of the present invention will be given in detail.

However, for reference numerals, with respect to the same elements, even though they may be displayed in different drawings, such elements use same reference numerals as much as possible. Also, in explaining the example embodiments, detailed description on known elements or functions will be omitted if it is determined that such description will interfere with understanding of the embodiments.

As shown, a vulvar sterilization dryer for women according to the present invention is configured to blow outside air or hot air toward the anal area, while irradiating the vulva with LED light sources having sterilization functions of removing harmful bacteria to thus prevent the occurrence of diseases, so that it is easy to sterilize and dry the vulva.

In specific, a vulvar sterilization dryer 100 for women according to the present invention includes a curved plate-like base member 110 adapted to cover a user's genital area and anus and having an anus spray hole 110a formed on one side thereof, a controller 120 disposed on one side of one surface of the base member 110 and having an operating button 121, a power supply part 122, and a PCB 123 adapted to control operations thereof, an irradiation part 130 fixedly disposed onto the other surface of the base member 110 in such a way as to operate controllable in wavelength under the control of the controller 120 to allow light emitted from LEDs 132 to be irradiated onto the genital area and the anus, an anus spray part 140 disposed close to the controller 120 on one surface of the base member 110 in such a way as to operate under the control of the controller 120 to allow outside air or hot air to be sprayed into the anal area through the anus spray hole 110a, and a covering member 150 configured to allow one end thereof to be detachably coupled to one surface of the base member 110 to coveringly protect the controller 120 and the anus spray part 140 from the outside.

Further, the vulvar sterilization dryer 100 includes fixing parts 160 located on one surface and the other surface of the base member 110, respectively, to allow the vulvar sterilization dryer 100 to be worn on the user's body.

Besides, the vulvar sterilization dryer 100 includes a cover pad 170 made of silicone and configured to allow one end thereof to be detachably attached to the other surface edge of the base member 110, while the other end thereof is protruding toward one direction, and the cover pad 170 serves to cover the user's skin when the vulvar sterilization dryer 100 is worn on the user's body.

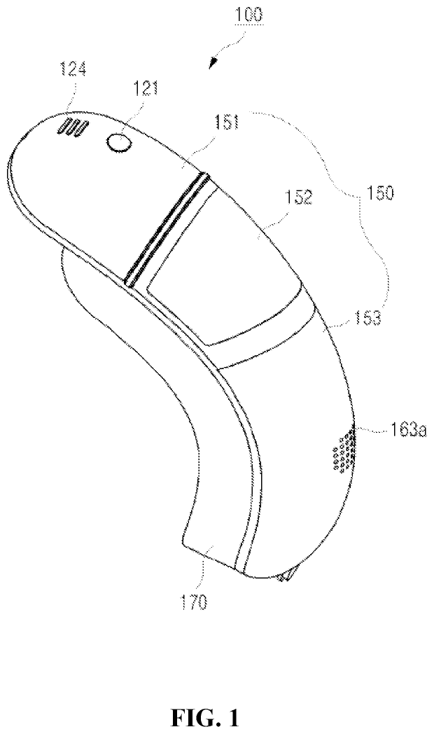

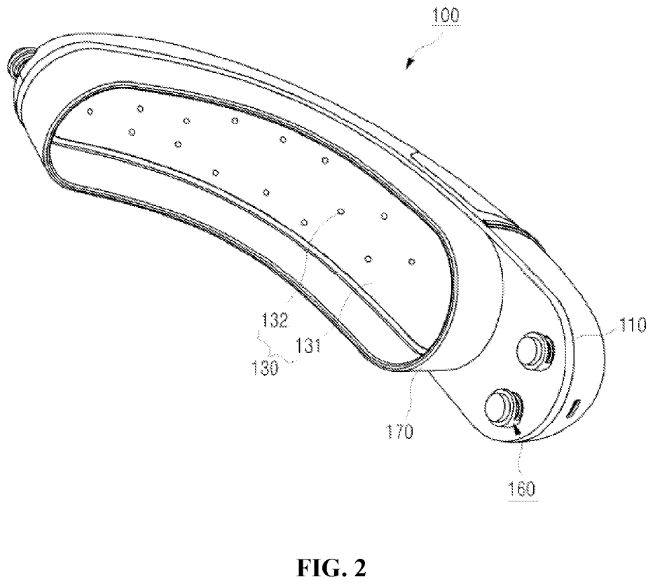

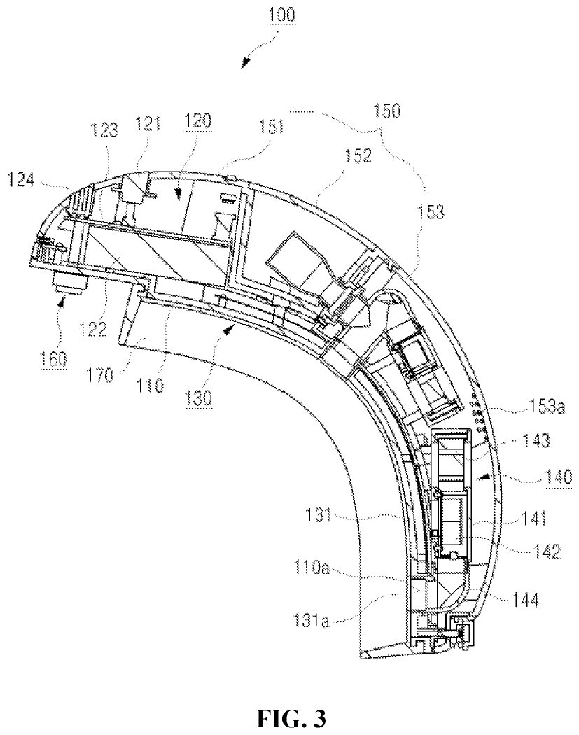

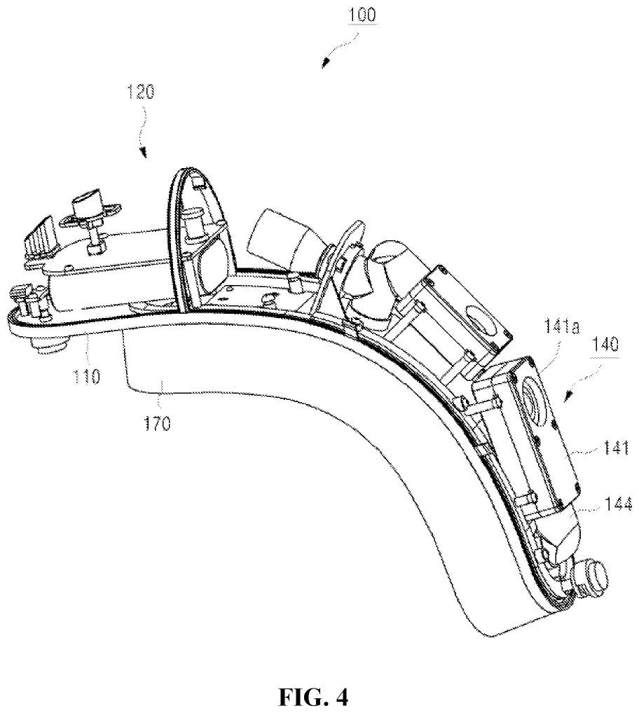

Hereinafter, the vulvar sterilization dryer 100 for women according to the present invention will be explained in detail with reference to FIGS. 1 to 4.

First, the vulvar sterilization dryer 100 according to the present invention is provided with the base member 110 that has the shape of an oval-shaped curved plate for covering the user's genital area and anus.

Further, the base member 110 has a covering member accommodation groove (having no reference numeral) and a cover pad accommodation groove (having no reference numeral) formed on the edges of one surface and the other surface thereof to insert one end of the covering member 150 and one end of the cover pad 170, respectively, so that the covering member 150 and the cover pad 170 are detachably mounted thereon.

Furthermore, the base member 110 is provided with the anus spray hole 110a formed thereon to spray the air supplied from the anus spray part 140 toward the anal area.

The controller 120 is disposed on one side of one surface of the base member 110 and has the operating button 121, the power supply part 122, and the PCB 123 in such a way as to control the operations of the anus spray part 140, that is, the operations of an anus heater 142 of the anus spray part 140 to allow the outside air or hot air to be sprayed into the anal area.

Further, the controller 120 includes notifying lamps 124 for emitting different light in such a way as to notify the user of charged amounts of the power supply part 122.

The irradiation part 130 is fixedly disposed onto the other surface of the base member 110 in such a way as to be controllable in wavelength under the control of the controller 120 to allow the light emitted from the LEDs 132 to be irradiated onto the genital area and the anus.

That is, the irradiation part 130 includes a curved plate-shaped substrate 131 having an anus communication hole 131a formed thereon in such a way as to communicate with the anus spray hole 110a, when fixed to the other surface of the base member 110, and the LEDs 132 located on the front surface of the substrate 131 in such a way as to emit light having a sterilization function of removing harmful bacteria to prevent occurrence of diseases toward the genital area and the anus.

In this case, the LEDs 132 are controllable in wavelength under the control of the controller 120.

The anus spray part 140 is fixedly disposed onto one surface of the base member 110 and operates under the control of the controller 120 to allow the outside air or hot air to be sprayed into the anal area through the anus spray hole 110a.

That is, the anus spray part 140 includes an anus spray housing 141 fixedly disposed onto one surface of the base member 110, an anus heater 142 located at one side of the interior of the anus spray housing 141 in such a way as to emit heat under the control of the controller 120, an anus blower 143 disposed close to the anus heater 142 at the inside of the anus spray housing 141 in such a way as to blow air toward the anus heater 142 under the control of the controller 120, and an anus supply pipe 144 having one end connected to the anus spray housing 141 and the other end connected to the anus spray hole 110a to allow the outside air or hot air to be supplied to the anus spray hole 110a.

Further, the anus spray housing 141 has an introduction hole 141a formed on top thereof in such a way as to introduce the outside air thereinto.

The covering member 150 is configured to allow one end thereof to be detachably coupled to one surface of the base member 110 to coveringly protect the controller 120 and the anus spray part 140 from the outside, and to do this, the covering member 150 consists of first, second, and third covering members 151, 152, and 153.

In this case, the first covering member 151 of the covering member 150 has a button exposing hole (having no reference numeral) and lamp exposure holes (having no reference numerals) formed thereon in such a way as to expose the operating button 121 and the notifying lamps 124 to the outside, and the third covering member 153 has introduction holes 153a formed thereon in such a way as to introduce the outside air thereinto.

The fixing parts 160 are fixedly located on one surface and the other surface of the base member 110, respectively, to allow the vulvar sterilization dryer 100 to be worn on the user's body.

That is, the fixing parts 160 consist of a belt (not shown) having magnets spaced apart from each other and fixed magnets (having no reference numerals) fixed to the rear end of one surface of the base member 110 and to the front side of the other surface of the base member 110 and having different poles from the magnets fixed to the belt so that the fixed magnets are attached to the magnets of the belt.

As mentioned above, the fixing parts 160 consist of the belt having the magnets and the fixed magnets fixed to the rear end of one surface and the front side of the other surface of the base member 110, but the present invention may not be limited thereto. For example, snap buttons may replace the magnets and the fixed magnets.

If it is desired to wear the vulvar sterilization dryer onto the user's body and dry the anal area, as shown in FIGS. 1 to 4, the belt of the fixing parts 160 is first worn onto the user's waist, and next, the irradiation part 130 is located to cover the user's genital area and anus. After that, the fixed magnets fixed to the rear end of one surface and the front side of the other surface of the base member 110 are attached to the magnets fixed to the belt.

As a result, the vulvar sterilization dryer 100 is fixedly worn onto the user's body.

As the vulvar sterilization dryer 100 is worn onto the user's body by means of the fixing parts 160, the cover pad 180 made of silicone is brought into close contact with the user's skin.

In the state where such wearing is completed, the operating button 121 of the controller 120 is pressed to operate the irradiation part 130 and the anus spray part 140, so that the light of the LEDs 132 of the irradiation part 130 is irradiated onto the genital area and the anus, and the outside air is sprayed into the anal area.

That is, if the vulvar sterilization dryer 100 is turned on, the light of the LEDs 132 of the irradiation part 130 is irradiated onto the genital area and the anus, and further, as the anus blower 143 located at the inside of the anus spray housing 141 of the anus spray part 140 operates, the outside air is supplied to the anus supply pipe 144. Next, the outside air supplied to the anus supply pipe 144 is sprayed into the anal area through the anus spray hole 110a and the anus communication hole 131a.

In this case, as the operating button 121 is pressed, the LEDs 132 of the irradiation part 130 have different wavelengths.

If it is desired to spray hot air into the anal area, while the outside air is being sprayed into the anal area, an operating button for the anus heater 142 of the anus spray part 140 is pressed to allow the anus heater 142 to operate.

As a result, the hot air is sprayed into the anal area through the anus spray hole 110a and the anus communication hole 131a.

As described above, the vulvar sterilization dryer 100 according to the present invention can dry the anal area with the outside air or hot air supplied from the anus spray part 140, so that the anal area is kept fresh, without being sticky, after the use thereof, thereby preventing the occurrence of diseases.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above teachings. The present invention may be modified in various ways and may have several exemplary embodiments. It is therefore intended that the scope of the invention be limited not by this detailed description, but rather by the claims appended hereto.

### INDUSTRIAL APPLICABILITY OF THE INVENTION

According to the present invention, the vulvar sterilization dryer for women is configured to blow outside air or hot air toward the anal area, while irradiating the vulva with the LED light sources having sterilization functions of removing harmful bacteria to thus prevent the occurrence of diseases, so that advantageously, it is easy to sterilize and dry the vulva.

1. A vulvar sterilization dryer for women, comprising:
a curved plate-like base member (110) adapted to cover a user's genital area and anus and having an anus spray hole (110a) formed on one side thereof;
a controller (120) disposed on one side of one surface of the base member (110) and having an operating button (121), a power supply part (122), and a PCB (123) adapted to control operations thereof;
an irradiation part (130) fixedly disposed onto the other surface of the base member (110) in such a way as to operate controllable in wavelength under the control of the controller (120) to allow light emitted from LEDs (132) to be irradiated onto the genital area and the anus;
an anus spray part (140) disposed close to the controller (120) on one surface of the base member (110) in such a way as to operate under the control of the controller (120) to allow outside air or hot air to be sprayed into the anal area through the anus spray hole (110a); and
a covering member (150) configured to allow one end thereof to be detachably coupled to one surface of the base member (110) to coveringly protect the controller (120) and the anus spray part (140) from the outside.

2. The vulvar sterilization dryer according to claim 1, wherein the irradiation part (130) comprises a curved plate-shaped substrate (131) having an anus communication hole (131a) formed thereon in such a way as to communicate with the anus spray hole (110a), when fixed to the other surface of the base member (110), and the LEDs (132) located on the front surface of the substrate (131) in such a way as to emit light having a sterilization function of removing harmful bacteria to prevent occurrence of diseases toward the genital area and the anus.

3. The vulvar sterilization dryer according to claim 1, wherein the anus spray part (140) comprises an anus spray housing (141) fixedly disposed onto one surface of the base member (110), an anus heater (142) located at one side of the interior of the anus spray housing (141) in such a way as to emit heat under the control of the controller (120), an anus blower (143) disposed close to the anus heater (142) at the inside of the anus spray housing (141) in such a way as to blow air toward the anus heater (142) under the control of the controller (120), and an anus supply pipe (144) having one end connected to the anus spray housing (141) and the other end connected to the anus spray hole (110a) to allow the outside air or hot air to be supplied to the anus spray hole (110a).
